(19) United States
(12) Patent Application Publication
(10) Pub. No.: US 2025/0033968 A1
(43) Pub. Date: Jan. 30, 2025

(54) ECONOMICAL METHOD FOR MASS PRODUCTION OF BIS(FLUOROSULFONYL)IMIDE METAL SALT SOLUTION

(71) Applicant: EVS TECHNOLOGIES, INC., Daejeon (KR)

(72) Inventor: Kwang Min LIM, Yongin-si (KR)

(21) Appl. No.: 18/716,116

(22) PCT Filed: Sep. 1, 2022
(86) PCT No.: PCT/KR2022/013107

§ 371 Date: Jun. 3, 2024

(30) Foreign Application Priority Data
Dec. 7, 2021 (KR) 10-2021-0173649
Jun. 24, 2022 (KR) 10-2022-0077434

(51) Int. Cl. C01B 21/086

(52) U.S. Cl. CPC C01B 21/086

(57) ABSTRACT

The present invention relates to a production method for a bis(fluorosulfonyl)imide metal salt solution, and more specifically, relates to a production method for powder of bis(fluorosulfonyl)imide metal salts represented by Formula 2 by performing azeotropic distillation on an aqueous bis(fluorosulfonyl)imide metal salt solution represented by Formula 2, which is produced by reacting bis(fluorosulfonyl)imide represented by Formula 1 as a starting material with a powder-type metalized reagent.

By the production method according to the present invention, no waste is generated at all by directly reacting a bis(fluorosulfonyl)imide compound with a powder-type metalized reagent without using a solvent so that, unlike the prior art, bis(fluorosulfonyl)imide metal salts can be produced in an eco-friendly way. The production method according to the present invention is very economical since a bis(fluorosulfonyl)imide metal salt solution can be easily mass-produced with a high yield and high purity by performing rapid condensation using azeotropic distillation to remove a small amount of water within 10 weight % generated during the reaction. The production process is simple with respect to a production process for powder form, and has advantages in terms of excellent storage and usability.

### TECHNICAL FIELD

The present invention relates to a novel production method for a liquid bis(fluorosulfonyl)imide metal salt product, and more specifically, to an economical production method in which a product of bis(fluorosulfonyl)imide metal salts is mass-produced with high efficiency by reacting bis(fluorosulfonyl)imide or an ammonium bis(fluorosulfonyl)imide salt as a starting material with a powder-type metalized reagent and then carrying out condensation or condensation/drying.

### BACKGROUND ART

Recently, a need for a high-performance secondary battery has increased with the commercialization of various mobile devices, and a secondary battery with performance such as high output, high energy density, and high discharge voltage has become necessary with the commercialization of electric vehicles and hybrid electric vehicles and the development of electric storage devices.

In an electrolyte solution composition suitable for the secondary battery, the importance of lithium salts has emerged. Accordingly, a need to economically produce a lithium bis(fluorosulfonyl)imide salt has emerged.

The lithium bis(fluorosulfonyl)imide salt has advantages over other lithium salt compounds such as lithium hexafluorophosphate since it has advantages such as high thermal stability, high electrical conductivity, low corrosivity, excellent low-temperature performance, and efficient film formation on the positive electrode and the negative electrode.

Recently, electric vehicles have been in the spotlight as an eco-friendly means of transportation that replace existing internal combustion engine vehicles, and the related market has been rapidly expanding. Accordingly, a characteristic lithium secondary battery has become necessary, and in particular, the lithium bis(fluorosulfonyl)imide salt exhibits the demand performance required for a lithium secondary battery for an electric vehicle, and thus the commercial importance thereof has been growing.

On the other hand, due to various limitations, it is limited to make it uneasy to mass-produce a lithium bis(fluorosulfonyl)imide salt inexpensively and economically.

In general, commercial products of bis(fluorosulfonyl)imide metal salts have a powder form; however, it takes a long drying time to produce a powder, and for mass production, it takes a large investment cost for a drying room, a dehumidifying room, and the like, which need to be built on a large scale. In addition, there is a problem that moisture needs to be strictly managed in the storage of the metal salt powder and in the process of the production of the electrolyte solution.

Much research has been carried out on the production of such bis(fluorosulfonyl)imide metal salts, including lithium bis(fluorosulfonyl)imide. However, in such a case, there is still a need for a production method for bis(fluorosulfonyl)imide metal salts, which is capable of achieving mass production more economically and is eco-friendly while achieving easy storage.

### DISCLOSURE OF THE INVENTION

### Technical Problem

Accordingly, an object of the present invention is to provide a production method for mass production of bis(fluorosulfonyl)imide metal salts having various forms, which is economical and efficient while being eco-friendly.

In addition, another object of the present invention is to provide bis(fluorosulfonyl)imide metal salts having high purity which are produced by the above-described production method.

### Technical Solution

While studying a method for producing a liquid bis(fluorosulfonyl)imide metal salt solution more easily with high purity and a high yield through an eco-friendly and a simple process, the inventors of the present invention found that it is possible to easily mass-produce a solution of the bis(fluorosulfonyl)imide metal salt with high purity and a high yield by reacting bis(fluorosulfonyl)imide or an ammonium bis(fluorosulfonyl)imide as a starting material with a powder-form metalized reagent in the presence of a solvent and then efficiently removing water generated after producing a bis(fluorosulfonyl)imide metal salt, whereby the present invention has been completed.

This production method does not generate waste water since the metalized reagent is used in a metal powder form rather than an aqueous solution when producing the bis(fluorosulfonyl)imide metal salt. As a result, the bis(fluorosulfonyl)imide metal salt can be produced in a very eco-friendly manner, and it is subjected to a simple water removal process, which makes it possible to easily mass-produce a solution of the bis(fluorosulfonyl)imide metal salt having high purity and a high yield.

Accordingly, the present invention provides a production method for a bis(fluorosulfonyl)imide metal salt solution, comprising: (i) a step of reacting a bis(fluorosulfonyl)imide compound represented by Formula 1 with a powder-type metalized reagent in a presence of a solvent to produce an aqueous solution of bis(fluorosulfonyl)imide metal salts represented by Formula 2; and (ii) a step of condensing the aqueous solution of bis(fluorosulfonyl)imide metal salts to produce a solution of the bis(fluorosulfonyl)imide metal salts represented by Formula 2.

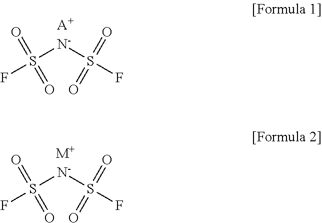

In Formula 1, A is H or NR¹R²R³R⁴, where R¹ to R⁴ may be the same or different from each other and is H or a C1 to C20 straight or branched alkyl group, and in Formula 2, M is lithium, sodium, or potassium.

In addition, the present invention provides a bis(fluorosulfonyl)imide metal salt solution having a purity of 99% or more, which is produced by the above-described production method.

### Advantageous Effects

In the production method for a bis(fluorosulfonyl)imide metal salt solution according to the present invention, no waste is generated at all by reacting a bis(fluorosulfonyl)imide compound with a powder-type metalized reagent so that, unlike the prior art, a bis(fluorosulfonyl)imide metal salt solution can be produced in an eco-friendly way.

In addition, while being very simple, this production method is very economical since a solution of bis(fluorosulfonyl)imide metal salts can be easily mass-produced with high yields and high purity by performing rapid condensation using azeotropic distillation to remove a small amount of water within 10 weight % generated during the reaction. In addition, this production method has a simple production process compared to a production process for a powder form, and it has excellent storage and usability. Therefore, the bis(fluorosulfonyl)imide metal salt solution produced according to the present invention can be widely applied to secondary batteries in electric vehicles (EV), power tools, and the like, which require high output, or antistatic agents.

### MODE FOR CARRYING OUT THE INVENTION

Hereinafter, the present invention will be described in more detail.

First, examining the technology in the prior art for making bis(fluorosulfonyl)imide metal salts, the production is such that a reaction is allowed to proceed to a solution form of bis(fluorosulfonyl)imide metal salts using various methods, a solvent is evaporated to create a condensed solution, a second solvent is added thereto to induce crystallization of the bis(fluorosulfonyl)imide metal salts, and the obtained crystals are filtered out, recovered, and dried. In most cases, an organic solution of bis(fluorosulfonyl)imide or an ammonium salt of bis(fluorosulfonyl)imide is brought into contact with an aqueous metalized solution to produce a solution form of bis(fluorosulfonyl)imide metal salts.

In most cases, bis(fluorosulfonyl)imide metal salts have an unusually high affinity for a solvent or water, and thus a part of bis(fluorosulfonyl)imide metal salts are lost by being dissolved in water in a case where the bis(fluorosulfonyl)imide metal salts are commercialized using the above-described method. In addition, they are also dissolved in a solvent during the crystallization process, the yield decreases inevitably, and thus the purity of the crystallized product is not high either. This is because a part of a pure bis(fluorosulfonyl)imide metal salt product is lost by being dissolved in a solvent or water. In addition, since it is inevitable to use, in the crystallization process, a second solvent that is insoluble in the product, a solvent mixture is generated as waste, and the waste water originating from the aqueous solution of the metalized reagent is also generated.

As described above, during this process, a small amount of water is not removed due to having a very high affinity for the bis(fluorosulfonyl)imide metal salt. On the other hand, although a product should be produced in an anhydrous state of less than 100 ppm due to features of the product as a substance for a secondary battery, a small amount of water is not easily removed using a conventional method, and the purity of the product may decrease due to the generation of impurities during the removal process through a process of, for example, raising the temperature for a long period of time. In addition, although thionyl chloride or the like is used to remove a small amount of water, the use thereof is not desirable at all since hydrochloric acid gas and SOₓ gas are generated inevitably, thereby for example, increasing the acidity in the product, raising the content of impurities such as Cl⁻ and SO₄²⁻, and causing deterioration of product quality. In addition, the condensation of water under reduced pressure by using a general method requires a high temperature of 60° C. or higher, a high vacuum of 1.0 mmHg or lower, and a long period of time of 24 to 48 hours, and thus it is not economical at all and is not suitable for commercial production.

On the other hand, the inventors of the present invention found that a bis(fluorosulfonyl)imide metal salt can be produced with high purity through a liquid-solid phase reaction or a solid-solid phase reaction by bringing bis(fluorosulfonyl)imide or an ammonium salt of bis(fluorosulfonyl)imide into contact with a metalized. As a representative example for description, in a case where the metal is lithium and the solidification reagent is the most common lithium hydroxide monohydrate, lithium bis(fluorosulfonyl)imide can be produced with a high yield and high purity. In a case where the purity, color, and impurity content were analyzed after removing the insoluble matter through simple filtration, all of them reached a satisfactory level. The key point was whether approximately 10% by weight of water could be removed to a product standard of 100 ppm or less, desirably 50 ppm or less, in a short period of time without deteriorating product purity.

Therefore, while studying various methods, the inventors of the present invention used a useful solvent that can cause azeotropic distillation together with water to check whether it is possible to remove approximately 10% by weight of water in a bis(fluorosulfonyl)imide metal salt reactant through azeotropic distillation. In this case, in a case where a solvent that mixes with bis(fluorosulfonyl)imide metal salts is used as an azeotropic distillation solvent, commercialization is possible as a solution form of the bis(fluorosulfonyl)imide metal salts after removing the water and simply filtering out the insoluble matter. In a case where a solvent that does not mix with bis(fluorosulfonyl)imide metal salts is used as an azeotropic distillation solvent, water is also removed, and the bis(fluorosulfonyl)imide metal salt product is crystallized and precipitated, which enables commercialization through easy filtering and a drying process.

Through such a simple process, both a powder product and a solution product of bis(fluorosulfonyl)imide metal salts can be produced. In particular, a solution product of bis(fluorosulfonyl)imide metal salts has a form that has not been commercialized to date, and this product, which is a secondary battery electrolyte, has several advantages.

That is to say, the bis(fluorosulfonyl)imide metal salt has strong hygroscopicity and agglomeration properties. Therefore, in a case where it is a powder, caution should be given to sealing, and moisture should be strictly excluded even during storage. In addition, even when being added to make an electrolyte solution, there are such inconveniences in use that moisture should be blocked as much as possible In fact, for this reason, it is packaged and used in a small package of about 5 kg.

On the other hand, in a case where the solvent in the solution product of bis(fluorosulfonyl)imide metal salts is a solvent that is used in a composition of an electrolyte solution, it is much free from moisturization and very convenient since it is possible to carry out the free addition of a fixed amount of liquid without the risk of moisturization. On the other hand, because there is a case where a powder product should be used, it is very important to develop a process that makes it possible to freely produce a powder and a solution product. Surprisingly, it was confirmed that water can be easily separated and removed in a short period of time by using azeotropic distillation, and thus it is possible to produce a high-purity lithium bis(fluorosulfonyl)imide product having a moisture content of 100 ppm or less without deteriorating purity. As a result of analyzing the purity, color, and impurity content of the lithium bis(fluorosulfonyl)imide product produced in this way, all of them reached a satisfactory level.

Through this, it was confirmed that a bis(fluorosulfonyl)imide metal salt can be easily synthesized industrially in large quantities. In the related art, the metalized reagent was used in a form of an aqueous solution in the metalization step, and thus it was inevitable to use excessive water, which inevitably resulted in the generation of waste water. On the other hand, in the present invention, a powder-type metalized reagent was used, and thus water was not used, thereby eliminating the generation of waste water.

In addition, it found that although in the related art, the bis(fluorosulfonyl)imide metal salt is commercialized by condensing it with a difficult method in an organic solvent, producing a product from the condensate through crystallization and carrying out separation by filtration and then drying, thereby undergoing four steps of condensation, production, separation, and drying, through which economic feasibility and workability are reduced due to an increase in work time, decreases in purity and yield, waste generation, and an increase in process cost, the development process by the inventors of the present invention is significantly excellent as compared with the existing process in terms of purity, yield, environment, and economic feasibility while carrying out the above-described processes at one time, whereby the present invention was completed.

Hereinafter, the present invention will be described in more detail as an embodiment as follows.

The present invention provides a method for preparing for a bis(fluorosulfonyl)imide metal salt solution, comprising: (i) a step of reacting a bis(fluorosulfonyl)imide compound represented by Formula 1 with a powder-type metalized reagent in the presence of a solvent to produce an aqueous solution of bis(fluorosulfonyl)imide metal salts represented by Formula 2; and (ii) a step of condensing the aqueous solution of bis(fluorosulfonyl)imide metal salts to produce a solution of the bis(fluorosulfonyl)imide metal salts represented by Formula 2.

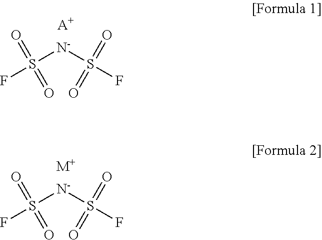

In Formula 1, A is H or NR¹R²R³R⁴, where R¹ to R⁴ may be the same or different from each other and is H or a C1 to C20 straight or branched alkyl group, and in Formula 2, M is lithium, sodium, or potassium.

In the present invention, the bis(fluorosulfonyl)imide metal salts represented by Formula 2 can be produced according to Reaction Formula 1 below:

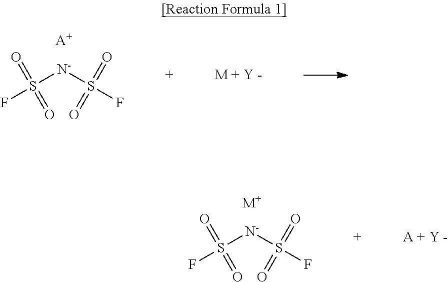

First, the step (i) is a step of producing an aqueous solution of bis(fluorosulfonyl)imide metal salts by reacting the bis(fluorosulfonyl)imide compound with a powder-type metalized reagent.

In the present invention, it is desirable to use a metal salt powder as a metalized reagent for metalization. In the related art, a metal salt powder was produced in a form of an aqueous solution and then used. However, the use thereof in such a form of an aqueous solution diluted the concentration of the reaction solution and thus had a limitation in that the loss of the bis(fluorosulfonyl)imide metal salts into the water layer had to be blocked. However, the method for preparing according to the present invention uses the metal salt powder as it is to allow a reaction to proceed, and thus it makes possible to carry out the reaction at high concentration, which is advantageous for mass production, and it is very eco-friendly since unnecessary waste water is generated. This point is different from the method for preparings in the related art.

Specifically, the bis(fluorosulfonyl)imide compound may include one or more selected from the group consisting of ammonium bis(fluorosulfonyl)imide, an alkylammonium bis(fluorosulfonyl)imide, a dialkylammonium bis(fluorosulfonyl)imide, a trialkylammonium bis(fluorosulfonyl)imide, bis(fluorosulfonyl)imide and a tetraalkylammonium bis(fluorosulfonyl)imide.

Specifically, the metalized reagent may be one selected from the group consisting of a hydroxide salt, a carbonate salt, and a bicarbonate salt of a metal selected from lithium, sodium, or potassium; however, the metalized reagent is not limited thereto. In this case, the metalized reagent may be used at an equivalent ratio of 1.0 to 1.1 with respect to bis(fluorosulfonyl)imide; however, the equivalent ratio thereof is not limited thereto.

Depending on the metalized reagent that is used in the step (i), 1 to 2 equivalents of water are generated, which is water that is generated as result a of a reaction between bis(fluorosulfonyl)imide and the metalized reagent. For example, in a case where the metalized reagent is sodium hydroxide, sodium bis(fluorosulfonyl)imide is synthesized and 1 equivalent of water is also generated together, as can be seen in Reaction Formula 1 above. In a case where the metalized reagent is lithium hydroxide monohydrate, regarding 1 equivalent of water as well, lithium bis(fluorosulfonyl)imide is synthesized through a reaction between bis(fluorosulfonyl)imide and lithium hydroxide, while generating together 1 equivalent of water, and 1 equivalent of water is generated from lithium hydroxide monohydrate. As a result, in this case, 2 equivalents of water are generated.

In the step (i), the reaction temperature is desirably-30° C. to 100° C., and the reaction time is desirably 0.5 to 4 hours. In a case where the reaction temperature is less than −30° C., the reaction rate slows down, and in a case where it exceeds 100° C., there is a disadvantage in that the impurity content increases.

In a case where a solvent is used in the step (i), a solvent that is capable of being used is any one or more selected among any one of ethers selected from the group consisting of diethyl ether, diisopropyl ether, methyl-t-butyl ether, and the like; alkoxyethanes such as dimethoxyethane and diethoxyethane; any one of esters selected from the group consisting of methyl acetate, ethyl acetate, propyl acetate, butyl acetate, and the like; any one of nitriles selected from the group consisting of acetonitrile, propionitrile, butyronitrile, and the like; any one of nitroalkanes selected from the group consisting of nitromethane, nitroethane, nitropropane, nitrobutane, and the like; any one of hydrocarbons selected from the group consisting of pentane, hexane, heptane, and the like; aromatic compounds such as benzene, toluene, and xylene; any one of alcohols selected from the group consisting of methanol, ethanol, propanol, butanol, and the like; any one of ketones selected from the group consisting of acetone, methyl ethyl ketone, methyl isopropyl ketone; and dialkyl carbonates selected from the group consisting of dimethyl carbonate, diethyl carbonate, ethylmethyl carbonate, and the like; as well as ethylene carbonate, propylene carbonate, and butylene carbonate.

In the step (i), a concentration of the bis(fluorosulfonyl)imide metal salts in the aqueous solution of bis(fluorosulfonyl)imide metal salts may be 0.1% by weight to 95.0% by weight.

In addition, a step of filtering and removing insoluble matter from the product produced after the step (i) may be carried out; however, such purification or filtration process is not necessarily carried out.

Next, the step (ii) is a step of subjecting the product containing a small amount of moisture in (i) described above to quick dehydration and drying of the water generated by using suitable drying equipment, thereby producing a bis(fluorosulfonyl)imide metal salt solution.

In a case where a solvent is used in the step (i), a solvent that is capable of being used is any one or more selected among any one of ethers selected from the group consisting of diethyl ether, diisopropyl ether, methyl-t-butyl ether, and the like; alkoxyethanes such as dimethoxyethane and diethoxyethane; any one of esters selected from the group consisting of methyl acetate, ethyl acetate, propyl acetate, butyl acetate, and the like; any one of nitriles selected from the group consisting of acetonitrile, propionitrile, butyronitrile, and the like; any one of nitroalkanes selected from the group consisting of nitromethane, nitroethane, nitropropane, nitrobutane, and the like; any one of hydrocarbons selected from the group consisting of pentane, hexane, heptane, and the like; aromatic compounds such as benzene, toluene, and xylene; any one of alcohols selected from the group consisting of methanol, ethanol, propanol, butanol, and the like; any one of ketones selected from the group consisting of acetone, methyl ethyl ketone, methyl isopropyl ketone; and dialkyl carbonates selected from the group consisting of dimethyl carbonate, diethyl carbonate, ethylmethyl carbonate, and the like; as well as ethylene carbonate, propylene carbonate, and butylene carbonate.

In the dehydration, it is possible to remove approximately 10% by weight of water in a bis(fluorosulfonyl)imide metal salt reactant through azeotropic distillation, by using a solvent that can cause azeotropic distillation together with water. The azeotropic distillation can be allowed to proceed at normal pressure or reduced pressure, and in this case, in a case where a solvent that mixes with bis(fluorosulfonyl)imide metal salts is used as an azeotropic distillation solvent, commercialization is possible as a solution form of the bis(fluorosulfonyl)imide metal salts after removing the water and simply filtering out the insoluble matter. In a case where a solvent that does not mix with bis(fluorosulfonyl)imide metal salts is used as an azeotropic distillation solvent, water is also removed, and the bis(fluorosulfonyl)imide metal salt product is crystallized and precipitated, which enables commercialization in a powder type through easy filtering and a drying process.

Through such a simple process, a solution product of bis(fluorosulfonyl)imide metal salts can be produced.

In a case of a solution product, additional drying can be carried out as necessary to remove moisture using additionally a general molecular sieve, and selective moisture removal is possible without product loss by using a product having a core size of 3 to 4 angstroms.

In addition, 5 to 20 parts by weight of a molecular sieve is added based on 100 parts by weight of the bis(fluorosulfonyl)imide solution, whereby it is possible to efficiently remove moisture from the product.

In addition, the present invention provides a bis(fluorosulfonyl)imide metal salt solution that is produced by the above-described production method and has a high purity of 99% or more. The bis(fluorosulfonyl)imide metal salt solution produced according to the present invention has high purity and extremely low impurities and thus can improve the electrochemical properties, output, and life span characteristics of a secondary battery in a case of being used as a metal salt in an electrolyte solution for a secondary battery.

In addition, the bis(fluorosulfonyl)imide metal salt solution is a solution containing 1 to 30 ppm of moisture, which is easy to be stored due to low stress on moisture content control and which makes it possible to omit a process of dissolving bis(fluorosulfonyl)imide metal salts in a solvent to produce an electrolyte for a secondary battery, which gives an advantage of excellent usability.

Hereinafter, the new method for producing bis(fluorosulfonyl)imide metal salts according to the present invention will be described in more detail through Examples below. However, the present invention is not limited to these Examples.

### Example 1. Production of Lithium Bis(Fluorosulfonyl)Imide Solution Using Ammonium Bis(Fluorosulfonyl)Imide and Lithium Hydroxide Monohydrate Powder

In a 1,000 mL container equipped with a stirring device, a Dean-Stark Trap, a condenser, and a thermometer, 297.3 g of ethylmethyl carbonate was added and dissolved in 99.1 g of ammonium bis(fluorosulfonyl)imide at 20° C., 21.0 g of lithium hydroxide monohydrate was slowly added thereto at 0° C., and the temperature was gradually raised to 20° C. while stirring until the reaction was completed.

Thereafter, the insoluble matter was filtered through filter paper, and a lithium bis(fluorosulfonyl)imide solution as a transparent liquid was subjected to reflux under a reduced pressure of 100 torr to obtain water at the top of the Dean-Stark Trap. After carrying out the reflux until the amount of water obtained reached the theoretical amount, the reactant was slowly cooled to room temperature. Thereafter, the insoluble matter was filtered through filter paper, and 388.0 g of a lithium bis(fluorosulfonyl)imide solution as a transparent liquid was obtained (yield: 97%, purity: 99.4%, acidity: 4 ppm, Cl⁻: 0.6 ppm, SO₄²⁻: 1.0 ppm, moisture: 28 ppm).

### Example 2. Production of Sodium Bis(Fluorosulfonyl)Imide Solution Using Ammonium Bis(Fluorosulfonyl)Imide and Sodium Hydroxide Powder

390.7 g of a sodium bis(fluorosulfonyl)imide solution was obtained in the same method as in Example 1, except that 20.0 g of sodium hydroxide was used instead of 21.0 g of lithium hydroxide monohydrate yield: 95%, purity: 99.4%, acidity: 5 ppm, Cl⁻: 0.8 ppm, SO₄²⁻: 1.1 ppm, moisture: 25 ppm).

### Example 3. Production of a Potassium Bis(Fluorosulfonyl)Imide Solution Using Ammonium Bis(Fluorosulfonyl)Imide and Potassium Hydroxide Powder

400.3 g of a potassium bis(fluorosulfonyl)imide solution was obtained in the same method as in Example 1, except that 33.0 g of potassium hydroxide was used instead of 21.0 g of lithium hydroxide monohydrate yield: 948, purity: 99.5%, acidity: 5 ppm, Cl⁻: 0.7 ppm, SO₄²⁻: 1.0 ppm, moisture: 28 ppm).

### Example 4. Additional Drying of Lithium Bis(Fluorosulfonyl)Imide Solution

Under a nitrogen atmosphere, in a 1,000 mL container equipped with a stirring device and a thermometer, 38.8 g of a molecular sieve (angstrom standard: 4) was added at 20° C. to 388.0 g of the lithium bis(fluorosulfonyl)imide solution obtained in Example 1, and then stirred and dried.

After drying, the molecular sieve and insoluble matter were filtered through filter paper to obtain a lithium bis(fluorosulfonyl)imide solution, which was dried from the initial 28 ppm to 8 ppm when measuring the moisture of the obtained lithium bis(fluorosulfonyl)imide solution.

As described above, although the present invention has been described in terms of limited Examples, the present invention is not limited thereto, and it goes without saying that various modifications and modifications can be made by those skilled in the art to which the present invention pertains, within the technical idea of the present invention and the scope of the appended claims and their equivalents which are described later.

1. A production method for a bis(fluorosulfonyl)imide metal salt solution, comprising:
(i) a step of reacting a bis(fluorosulfonyl)imide compound represented by Formula 1 with a powder-form metalized reagent in a presence of a solvent to produce an aqueous solution of bis(fluorosulfonyl)imide metal salts represented by Formula 2; and
(ii) a step of condensing the aqueous solution of bis(fluorosulfonyl)imide metal salts to produce a solution of the bis(fluorosulfonyl)imide metal salts represented by Formula 2: 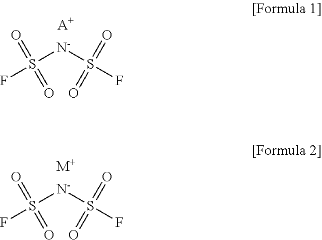
wherein, in Formula 1 above, A is H or NR¹R²R³R⁴, R¹ to R⁴ are the same or different from each other and are H or a C1 to C20 linear or branched alkyl group, and
in Formula 2 above, M is lithium, sodium, or potassium.

2. The production method according to claim 1, wherein the bis(fluorosulfonyl)imide compound includes one or more selected from the group consisting of bis(fluorosulfonyl)imide, ammonium bis(fluorosulfonyl)imide, an alkylammonium bis(fluorosulfonyl)imide, a dialkylammonium bis(fluorosulfonyl)imide, a trialkylammonium bis(fluorosulfonyl)imide, and a tetraalkylammonium bis(fluorosulfonyl)imide.

3. The production method according to claim 1, wherein the metalized reagent is one selected from the group consisting of a hydroxide salt, a carbonate salt, and a bicarbonate salt of a metal selected from lithium, sodium, or potassium.

4. The production method according to claim 1, wherein the metalized reagent is used at an equivalent ratio of 1.0 to 1.1 with respect to the bis(fluorosulfonyl)imide compound.

5. The production method according to claim 1, wherein the condensation in the step (ii) removes moisture using a solvent that is capable of being subjected to azeotropic distillation together with the moisture.

6. The production method according to claim 1, wherein the solvent is any one or more selected among any one of ethers selected from the group consisting of diethyl ether, diisopropyl ether, methyl-t-butyl ether, and the like; alkoxyethanes such as dimethoxyethane and diethoxyethane; any one of esters selected from the group consisting of methyl acetate, ethyl acetate, propyl acetate, butyl acetate, and the like; any one of nitriles selected from the group consisting of acetonitrile, propionitrile, butyronitrile, and the like; any one of nitroalkanes selected from the group consisting of nitromethane, nitroethane, nitropropane, nitrobutane, and the like; any one of hydrocarbons selected from the group consisting of pentane, hexane, heptane, and the like; aromatic compounds such as benzene, toluene, and xylene; any one of alcohols selected from the group consisting of methanol, ethanol, propanol, butanol, and the like; any one of ketones selected from the group consisting of acetone, methyl ethyl ketone, methyl isopropyl ketone; and dialkyl carbonates selected from the group consisting of dimethyl carbonate, diethyl carbonate, ethylmethyl carbonate, and the like; as well as ethylene carbonate, propylene carbonate, and butylene carbonate.

7. The production method according to claim 1, wherein in the step (i), a concentration of the bis(fluorosulfonyl)imide metal salts in the aqueous solution of bis(fluorosulfonyl)imide metal salts is 0.1% by weight to 95.0% by weight.

8. The production method according to claim 1,
wherein in the step (ii), a drying process is additionally performed, and
the drying is performed by passing through a molecular sieve.

9. The production method according to claim 8, wherein the molecular sieve has a core size of 2 to 5 angstroms.

10. The production method according to claim 8, wherein 5 to 20 parts by weight of the molecular sieve is added based on 100 parts by weight of a bis(fluorosulfonyl)imide solution.

11. A bis(fluorosulfonyl)imide metal salt solution having a purity of 99% or more, wherein the bis(fluorosulfonyl)imide metal salt solution is produced by the production method according to claim 1.

12. The bis(fluorosulfonyl)imide metal salt solution according to claim 11, wherein the bis(fluorosulfonyl)imide metal salt solution has a moisture content of 1 to 30 ppm.
